(19) United States
(12) Patent Application Publication
(10) Pub. No.: US 2010/0298636 A1
(43) Pub. Date: Nov. 25, 2010

(54) FLEXIBLE RIGIDIZING INSTRUMENTS

(76) Inventors: Salvatore Castro, Raleigh, NC (US); Michael Castro, Raleigh, NC (US)

Correspondence Address: SYNECOR LLC, P.O. BOX 5325, LARKSPUR, CA, 94977, US

(21) Appl. No.: 12/708,480

(22) Filed: Feb. 18, 2010

Related U.S. Application Data
(60) Provisional application No. 61/153,951, filed on Feb. 19, 2009.

(51) Int. Cl. A61B 1/00

(52) U.S. Cl. 600/104

(57) ABSTRACT

Medical instruments suitable for use in minimally invasive medical procedures have a first, highly flexible, state which allows the instruments to be easily passed into a body cavity through a trocar, cannula, catheter, or other access device positioned to give access into the body cavity. The instruments can then be arranged into a chosen shape, and rigidized in the chosen shape, allowing the user to use the instruments in a shape most suitable for the surrounding anatomy.

The present application claims the benefit of U.S. Provisional Application No. 61/153,951, filed 19 Feb. 2009.

### BACKGROUND

Flexible endoscopic surgical dissectors, staplers, retractors, graspers, forceps etc are conventional instruments used for endoscopic procedures. Advances in laparoscopic or single port surgical techniques have created new uses for flexible instruments beyond their traditional endoscopic use. Advanced access systems have given rise to the need for instrument capable of sufficiently flexibility to pass through bends in the access system, but also capable of transmitting sufficient axial forces for operation of an end effector to retract, grasp, compress, staple, or to otherwise impart forces to the tissue.

### BRIEF DESCRIPTION OF THE DRAWINGS

FIG. 1 is a perspective view of an embodiment of an instrument shaft of the type used for each of the instruments disclosed herein, with one segment shown removed from the shaft;

FIG. 2 is a distal perspective view of a segment from the shaft of FIG. 1;

FIG. 3 is a perspective view of the proximal side of the segment of FIG. 2;

FIG. 4 is a perspective view of a grasper using an instrument shaft of the type shown in FIG. 1;

FIG. 5 shows the distal end of the grasper of FIG. 4;

FIG. 6 shows the handle of the grasper of FIG. 4;

FIG. 7 is a perspective view of an endoscopic stapler using an instrument shaft of the type shown in FIG. 1;

FIG. 8 shows the distal end of the stapler of FIG. 7;

FIG. 9 shows the proximal end of the stapler of FIG. 7;

FIG. 10 is a perspective view of a retractor using an instrument shaft of the type shown in FIG. 1;

FIG. 11 shows the distal end of the retractor of FIG. 10;

FIG. 12 shows the proximal end of the retractor of FIG. 10.

### DETAILED DESCRIPTION

Disclosed are medical instruments suitable for use in minimally invasive medical procedures. These medical instruments have a first, highly flexible, state which allows the instruments to be easily passed into a body cavity through a trocar, cannula, catheter, or other access device positioned to give access into the body cavity. The instruments can then be arranged into a chosen shape, and rigidized in the chosen shape, allowing the user to use the instruments in a shape most suitable for the surrounding anatomy.

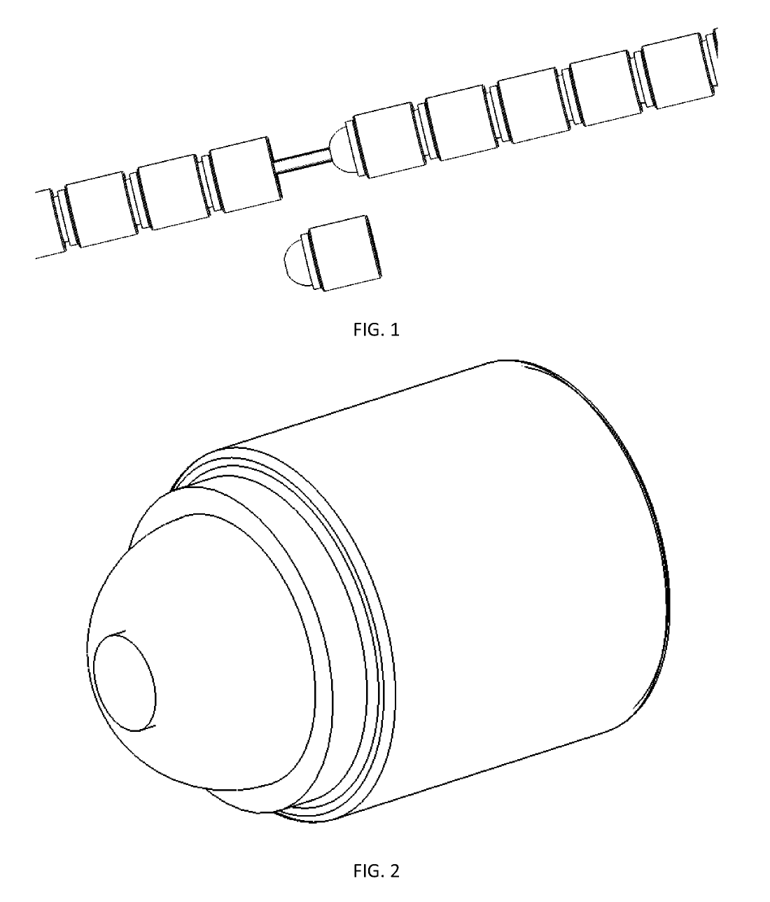

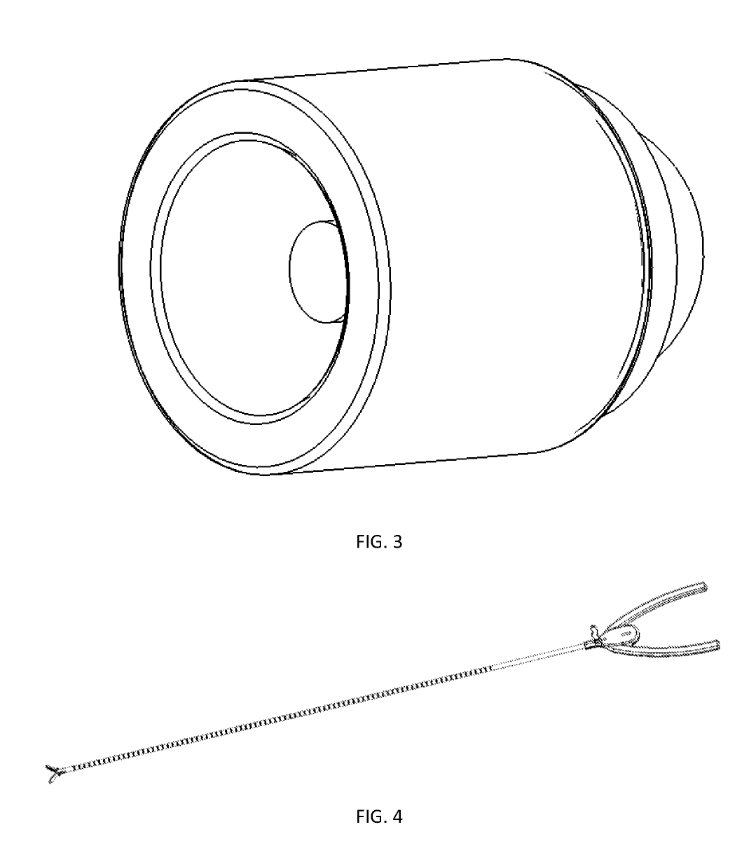

Each of the instruments disclosed herein includes an elongate shaft of the type shown in FIG. 1. The shaft is formed of a plurality of individual segments. Each segment includes a distal end comprising a semi- or partially-spherical convex surface (FIG. 2), and a proximal end including a receptacle (preferably having a partially or semi-spherical concave surface—FIG. 4) for receiving the convex surface. In the illustrated embodiments, the proximal end of the segment has a generally cylindrical configuration, although other shapes may be used. The longitudinal length of the proximal section (e.g. the length of the cylinder) may be chosen to give the shaft the desired amount of flexibility, with shorter proximal sections giving the greatest flexibility, and longer proximal sections giving less flexibility. Each segment has a central opening.

As can be seen in FIG. 1, the central opening of each segment is threaded over an elongate cable or tube. The segments are arranged on the cable such that each convex surface faces a corresponding concave surface. Given the semi-spherical shape of these surfaces, each interface between adjacent segments forms a sort of ball and socket joint, giving the segments the ability to move relative to one another with a large degree of freedom and making the shaft highly flexible.

In each instrument employing the shaft, the cable extends into a handle having an actuator. The actuator is coupled to the cable such that movement of the actuator from a first position to a second position pulls the cable proximally, thereby shortening the effective length of the cable available to be occupied by the segments. Each segment is thereby drawn into tight engagement with neighboring segments, with each convex surface nesting within the receptacle formed by the adjacent concave surface. The shaft is therefore converted to a rigid state and will retain the shape it possessed at the time the cable was tensioned. If desired, prior to tension of the cable, a separate instrument such a forceps or grasping device may be used to support a portion of the shaft so as to form the shaft into the shape desired for the shaft once it has been moved to its rigid state.

There are many types of instruments that may be made using the shaft of FIG. 1. Three such instruments will be described, although it should be appreciated that other embodiments are readily conceivable and the disclosed embodiments may be modified in various ways.

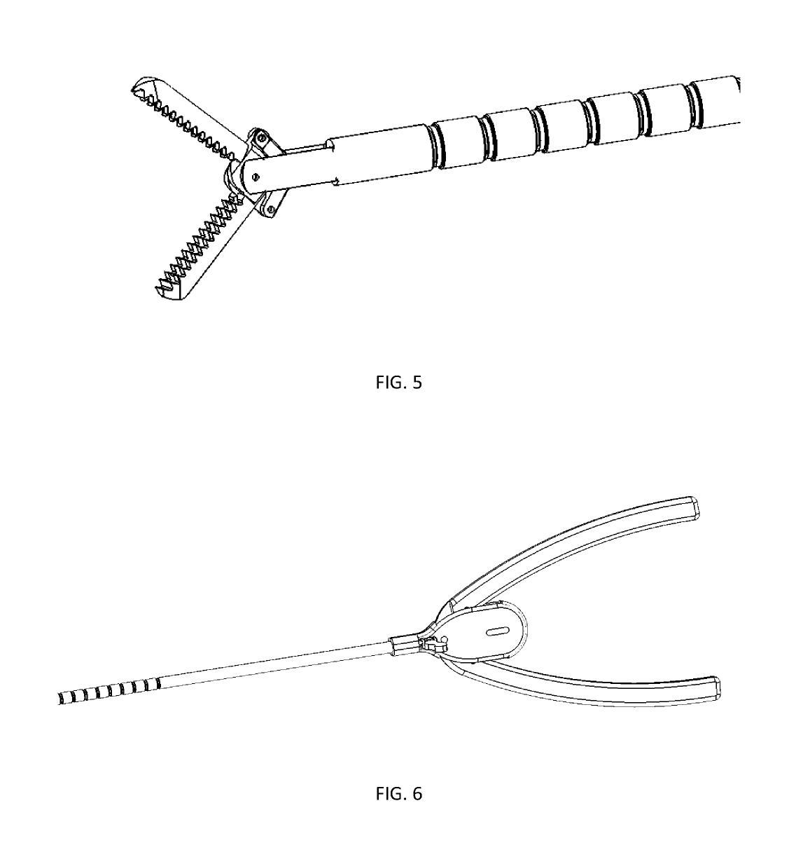

A first example is the grasper shown in FIG. 4. The grasper includes an end effector on its distal end (FIG. 5) and a handle on its proximal end (FIG. 6). The handle includes a first actuator, such as the illustrated lever on the distal portion of the handle, which is manipulatable to rigidize the shaft as described above. A second actuator, such as the pair of levers squeezable towards one another, is used to close the jaws of the grasper.

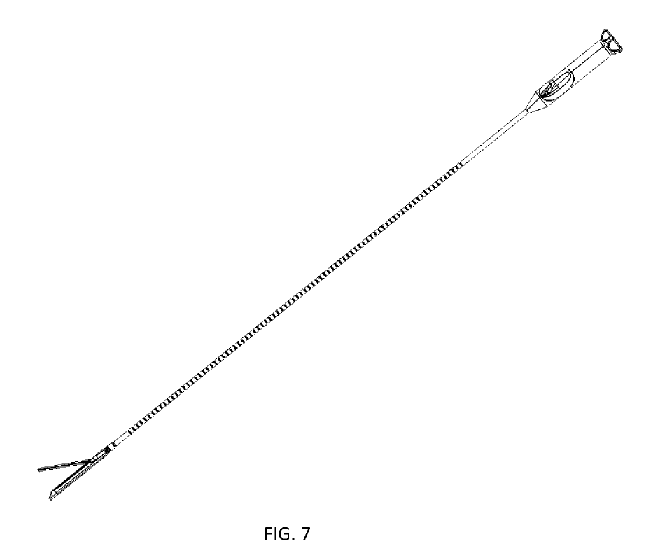

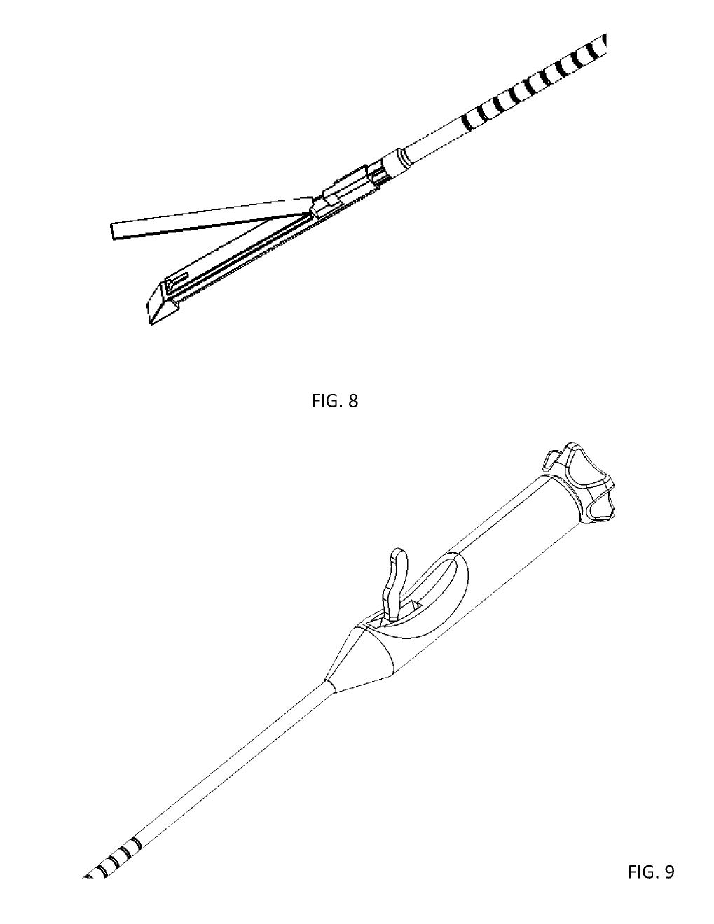

A second exemplary instrument is the endoscopic stapler, which performs compression, stapling and optional cutting, of the type shown in FIG. 7. At the distal end (FIG. 8) is a stapler head containing a staple cartridge containing an array of staples. An anvil is pivotable relative to the staple cartridge. A lever on the proximal end (FIG. 9) is used to rigidize the shaft. The lever or another actuator may be used to close the anvil against tissue positioned against the stapler head. A rotatable knob engages a pull-wire within the shaft. The pull-wire draws a shuttle through the staple cartridge to drive staples from the stapler head through tissue engaged between the stapler head and anvil. In this embodiment, the cable for rigidizing the shaft and the pull-wire for the stapler may extend side-by-side within the shaft, thereby minimizing longitudinal twisting of the shaft during use. In use, the shaft is rigidized, the jaws are manipulated to position the stapler head and anvil on opposite sides of the tissue, the tissue is compressed between the jaws, and the staples are then advanced through the tissue. A cutting element may then pass through the stapler head to divide the tissue between applied lines of staples.

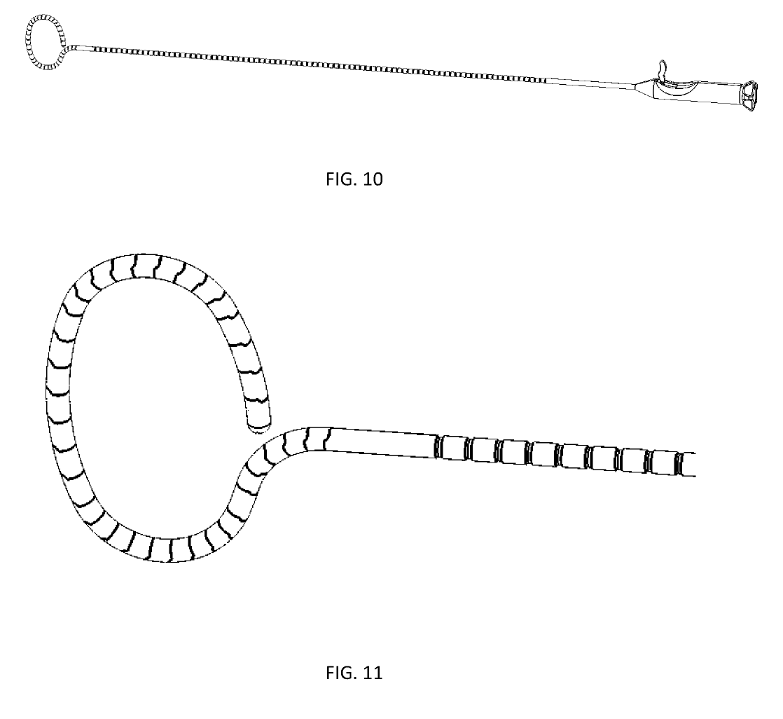

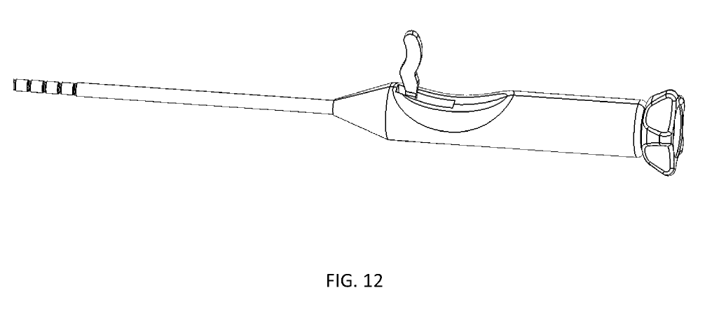

A third exemplary embodiment is the retractor device shown in FIG. 10. The distal end of the retractor has a retracting element is formed of a plurality of segments strung on a cable that extends through the shaft to the handle. These segments differ from the segments of the shaft. In particular, the segments forming the retracting element have proximal and distal surfaces oriented so that when they are pulled to a rigid position upon application of tension to their corresponding cable, they assemble into a predetermined shape, such as the loop shown in FIGS. 10 and 11. In the retractor embodiment, the handle includes a lever as described above for rigidizing the shaft, and a knob that may be rotated to tighten the cable and to thereby rigidize the loop. The rigidity achieved using this device is particularly suitable for large organ retraction, such as retraction of the liver during surgery on the gallbladder.

During use of the instruments disclosed herein, the shape into which the shaft will be rigidized may be selected by the user by moving the shaft (in its flexible form) into the shaft desired for the rigid form of the shaft. In an exemplary method, an instrument is passed into the body via a flexible cannula having a deflectable distal end. The flexible cannula is deflected (e.g. using pullwires) to position the shaft of the instrument in a desired shape, and the instrument is then rigidized to retain that shape.

Any and all applications referred to herein, including for purposes of priority, are hereby incorporated herein by reference.

We claim:

1. A medical instrument, comprising:
an elongate shaft including a first plurality of segments, each segment including a first end comprising a partially-spherical convex surface and a second end including a receptacle, the segments arranged such with the partially-spherical convex surfaces of a plurality of the segments disposed within the receptacles of adjacent segments;
an end effector coupled to the distalmost segment;
a least one cable extending through the end effector and the plurality of segments;
a handle having a first actuator for selectively applying and releasing tension on the cable, and a second actuator operatively coupled to the end effector for operation thereof, wherein the elongate shaft has a first, highly flexible state and a second, rigid, state upon actuation of the first actuator to tension to the cable.

2. The medical instrument of claim 1 wherein the shape of the shaft in the rigid shape is not predetermined.

3. The medical instrument of claim 2, wherein application of tension to the cable causes the shaft to become rigid while retaining the shape possessed by the shaft prior to the application of tension.

4. The medical instrument of claim 1, wherein the receptable includes a semi-spherical surface.

5. The medical instrument of claim 1, wherein at portion of each segment surrounding the receptacle is defined by a generally cylindrical wall.

6. The medical instrument of claim 2, wherein the end effector comprises a second plurality of segments positioned distally of the first plurality of segments, and wherein the medical instrument comprises a second cable extending through the end effector and the elongate shaft to the handle, the handle manipulatable to tension the second cable to move the second plurality of segments to a rigid state having a predetermined shape.

7. The medical instrument of claim 6, wherein the end effector is a retractor.

8. The medical instrument of claim 1, wherein the end effector includes pair of jaws.

9. The medical instrument of claim 8, wherein a first one of the jaws includes at least one anvil for forming a staple and a second one of the jaws includes at least one staple having legs advanceable into contact with the anvil.

10. A method of performing a procedure on body tissue, including the steps of:
introducing a instrument into a body cavity, the instrument including plurality of segments arranged to form an elongate shaft, and an end effector on the distal end of the shaft;
engaging an actuator to tension a cable in the shaft, drawing each segment into tight engagement with neighboring segments, thereby converting the shaft to a rigid state, causing the shaft retain the shape it possessed at the time the cable was tensioned;
and, after tensioning the cable, performing a procedure on the tissue using the end effector.

11. The method of claim 10, wherein each segment includes a first end having a convex surface and a second end having a concave surface, wherein tensioning the cable causes a plurality of the convex surfaces to tightly nest within the receptacle formed by the adjacent concave surfaces.

12. The method of claim 10, wherein the end effector comprises a second plurality of segments positioned distally of the first plurality of segments, and wherein the medical instrument comprises a second cable extending through the end effector and the elongate shaft to the handle, wherein the method further includes tensioning the second cable to move the second plurality of segments to a rigid state having a predetermined shape.

13. The method of claim 12 wherein the first and second cables are tensioned simultaneously.

14. The method of claim 12, further including the step of, after tensioning the second cable, retracting the tissue using the second plurality of segments.

15. The method of claim 12, further including the step of using the end effector to staple tissue.

16. The method of claim 12, further including the step of using the end effector to grasp tissue.
